(19) United States
(12) Patent Application Publication
(10) Pub. No.: US 2007/0093417 A1
(43) Pub. Date: Apr. 26, 2007

(54) Novel GLP-1 analogues linked to albumin-like agents

(75) Inventors: Thomas Kruse Hansen, Herlev (DK); Magali Zundel, Soborg (DK); Kjeld Madsen, Vaerlose (DK); Anne Svendsen, Vekso (DK); Christine Bruun Schiodt, Bronshoj (DK); Jesper Lau, Farum (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/454,348

(22) Filed: Jun. 16, 2006

Related U.S. Application Data
(63) Continuation of application No. PCT/DK04/00887, filed on Dec. 17, 2004.

(30) Foreign Application Priority Data
Dec. 18, 2003 (DK) PA 2003 01883

(51) Int. Cl. C07K 14/605

(52) U.S. Cl. 514/12; 530/399; 530/363

(57) ABSTRACT

Novel GLP-1 agonists which are protracted by coupling to a protraction protein.

### CROSS-REFERENCE TO RELATED PATENT APPLICATIONS

This patent application is a continuation of International Patent Application No. PCT/DK2004/000887 filed Dec. 17, 2004 and claims priority to U.S. provisional application No. 60/531,205 filed Dec. 19, 2003 and Danish Patent Application No. PA 2003 01883, filed Dec. 18, 2003.

### FIELD OF THE INVENTION

The present invention relates to novel GLP-1 compounds, to pharmaceutical compositions comprising these compounds and to the use of the compounds for the treatment of diseases related to diabetes.

### BACKGROUND OF THE INVENTION

Diabetes mellitus is a metabolic disorder in which the ability to utilize glucose is partly or completely lost. About 5% of all people suffer from diabetes and the disorder approaches epidemic proportions. Since the introduction of insulin in the 1920's, continuous efforts have been made to improve the treatment of diabetes mellitus.

One peptide expected to become very important in the treatment of diabetes is glucagon-like peptide-1 (GLP-1). Human GLP-1 is a 37 amino acid residue peptide originating from preproglucagon which is synthesized i.a. in the L-cells in the distal ileum, in the pancreas and in the brain. GLP-1 is an important gut hormone with regulatory function in glucose metabolism and gastrointestinal secretion and metabolism. GLP-1 stimulates insulin secretion in a glucose-dependant manner, stimulates insulin biosynthesis, promotes beta cell rescue, decreases glucagon secretion, gastric emptying and food intake. Human GLP-1 is hydrolysed to GLP-1 (7-37) and GLP-1 (7-36)-amide which are both insulinotropic peptides. A simple system is used to describe fragments and analogues of this peptide. Thus, for example, [Gly⁸]GLP-1 (7-37) designates an analogue of GLP-1 (7-37) formally derived from GLP-1 (7-37) by substituting the naturally occurring amino acid residue in position 8 (Ala) by Gly. Similarly, (N^{ε34}-tetradecanoyl)[Lys³⁴]GLP-1 (7-37) designates GLP-1 (7-37) wherein the ε-amino group of the Lys residue in position 34 has been tetradecanoylated. PCT publications WO 98/08871 and WO 99/43706 disclose stable derivatives of GLP-1 analogues, which have a lipophilic substituent. These stable derivatives of GLP-1 analogues have a protracted profile of action compared to the corresponding GLP-1 analogues.

In the last decade a number of peptides have been isolated from the venom of the Gila monster lizards (Heloderma suspectum and Heloderma horridum). Exendin-4 is a 39 amino acid residue peptide isolated from the venom of Heloderma suspectum, and this peptide shares 52% homology with GLP-1 (7-37) in the overlapping region. Exendin-4 is a potent GLP-1 receptor agonist which has been shown to stimulate insulin release and ensuing lowering of the blood glucose level when injected into dogs. The group of exendin-4(1-39), certain fragments thereof, analogs thereof and derivatives thereof, are potent insulinotropic agents. Most importantly the group of exendin-4(1-39), insulinotropic fragments thereof, insulinotropic analogs thereof and insulinotropic derivatives thereof.

A range of GLP-1 compounds including exendin compounds have been synthesized and studied in particular in relation the plasma half-life. Low plasma halflifes may be due to chemical stability towards peptidases (mainly dipeptidyl aminopeptidase IV) and to renal clearance. However, these variants of insulionotropic peptides have hitherto not showed protracted effects beyond what will suffice for at product to be administered to the patient once daily. A second generation GLP-1 compounds are needed which can be administered to the patients only once weekly or even less frequently.

U.S. Pat. No. 6,329,336 discloses the injection of highly reactive GLP-1 peptides into plasma, wherein chemical reactions will take place with blood components, such as serum albumin.

WO 02/46227 discloses fusion proteins between a GLP-1 compound and human serum albumin.

WO 2003/103572 discloses conjugates of GLP-1 analogs and a blood component.

It is an object of the present invention to provide GLP-1 analogues including exendin peptides linked to protein having a long half-life in human plasma, thereby facilitating a once-weekly treatment of patients. It is also an object of the present invention to provide GLP-1 peptides which are less prone to aggregation, a well known problem associated with the glucagon-like peptides. Being less prone to aggregation facilitates economical manufacturing processes as well as enabling the compounds to be administered by medical infusion pumps.

Definitions

In the present specification, the following terms have the indicated meaning

The term “polypeptide” and “peptide” as used herein means a compound composed of at least five constituent amino acids connected by peptide bonds. The constituent amino acids may be from the group of the amino acids encoded by the genetic code and they may natural amino acids which are not encoded by the genetic code, as well as synthetic amino acids. Natural amino acids which are not encoded by the genetic code are e.g. hydroxyproline, γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), β-alanine, 3-aminomethyl benzoic acid, anthranilic acid.

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. A simple system is often used to describe analogues: For example [Arg³⁴]GLP-1 (7-37)Lys designates a GLP-1 (7-37) analogue wherein the naturally occurring lysine at position 34 has been substituted with arginine and wherein a lysine has been added to the terminal amino acid residue, i.e. to the Gly³⁷. All amino acids for which the optical isomer is not stated is to be understood to mean the L-isomer.

The term “derivative” as used herein in relation to a peptide means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like. An example of a derivative of GLP-1(7-37) is N^{ε26}-((4S)-4-(hexadecanoylamino)-butanoyl)[Arg³⁴, Lys²⁶]GLP-1-(7-37).

The term “GLP-1 agonist” as used herein means a compound which stimulates the formation of cAMP in a suitable medium containing the human GLP-1 receptor. The potency of a GLP-1 agonist is determined by calculating the EC₅₀ value from the dose-response curve as described below.

Baby hamster kidney (BHK) cells expressing the cloned human GLP-1 receptor (BHK-467-12A) were grown in DMEM media with the addition of 100 IU/mL penicillin, 100 μg/mL streptomycin, 5% fetal calf serum and 0.5 mg/mL Geneticin G-418 (Life Technologies). The cells were washed twice in phosphate buffered saline and harvested with Versene. Plasma membranes were prepared from the cells by homogenisation with an Ultraturrax in buffer 1 (20 mM HEPES-Na, 10 mM EDTA, pH 7.4). The homogenate was centrifuged at 48,000×g for 15 min at 4° C. The pellet was suspended by homogenization in buffer 2 (20 mM HEPES-Na, 0.1 mM EDTA, pH 7.4), then centrifuged at 48,000×g for 15 min at 4° C. The washing procedure was repeated one more time. The final pellet was suspended in buffer 2 and used immediately for assays or stored at −80° C.

The functional receptor assay was carried out by measuring cyclic AMP (cAMP) as a response to stimulation by the insulinotropic agent. cAMP formed was quantified by the AlphaScreen™ cAMP Kit (Perkin Elmer Life Sciences). Incubations were carried out in half-area 96-well microtiter plates in a total volume of 50 μL buffer 3 (50 mM Tris-HCl, 5 mM HEPES, 10 mM MgCl₂, pH 7.4) and with the following additions: 1 mM ATP, 1 μM GTP, 0.5 mM 3-isobutyl-1-methylxanthine (IBMX), 0.01% Tween-20, 0.1% BSA, 6 μg membrane preparation, 15 μg/mL acceptor beads, 20 μg/mL donor beads preincubated with 6 nM biotinyl-cAMP. Compounds to be tested for agonist activity were dissolved and diluted in buffer 3. GTP was freshly prepared for each experiment. The plate was incubated in the dark with slow agitation for three hours at room temperature followed by counting in the Fusion™ instrument (Perkin Elmer Life Sciences). Concentration-response curves were plotted for the individual compounds and EC₅₀ values estimated using a four-parameter logistic model with Prism v. 4.0 (GraphPad, Carlsbad, Calif.).

The term “GLP-1 peptide” as used herein means GLP-1(7-37) (SEQ ID No 2), a GLP-1(7-37) analogue, a GLP-1 (7-37) derivative or a derivative of a GLP-1 (7-37) analogue. In one embodiment the GLP-1 peptide is an insulinotropic agent.

The term “exendin-4 peptide” as used herein means exendin-4(1-39) (SEQ ID No 3), an exendin-4(1-39) analogue, an exendin-4(1-39) derivative or a derivative of an exendin-4(1-39) analogue. In one embodiment the exendin-4 peptide is an insulinotropic agent.

The term “DPP-IV protected” as used herein referring to a polypeptide means a polypeptide which has been chemically modified in order to render said compound resistant to the plasma peptidase dipeptidyl aminopeptidase-4 (DPP-IV). The DPP-IV enzyme in plasma is known to be involved in the degradation of several peptide hormones, e.g. GLP-1, GLP-2, Exendin-4 etc. Thus, a considerable effort is being made to develop analogues and derivatives of the polypeptides susceptible to DPP-IV mediated hydrolysis in order to reduce the rate of degradation by DPP-IV. In one embodiment a DPP-IV protected peptide is more resistant to DPP-IV than GLP-1 (7-37) or Exendin-4(1-39).

Resistance of a peptide to degradation by dipeptidyl aminopeptidase IV is determined by the following degradation assay:

Aliquots of the peptide (5 nmol) are incubated at 37° C. with 1 μL of purified dipeptidyl aminopeptidase IV corresponding to an enzymatic activity of 5 mU for 10-180 minutes in 100 μL of 0.1 M triethylamine-HCl buffer, pH 7.4. Enzymatic reactions are terminated by the addition of 5 μL of 10% trifluoroacetic acid, and the peptide degradation products are separated and quantified using HPLC analysis. One method for performing this analysis is: The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 1999; 79:93-102 and Mentlein et al. Eur. J. Biochem. 1993; 214:829-35. Peptides and their degradation products may be monitored by their absorbance at 220 nm (peptide bonds) or 280 nm (aromatic amino acids), and are quantified by integration of their peak areas related to those of standards. The rate of hydrolysis of a peptide by dipeptidyl aminopeptidase IV is estimated at incubation times which result in less than 10% of the peptide being hydrolysed.

The term “C₁₋₆-alkyl” as used herein means a saturated, branched, straight or cyclic hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl, cyclohexane and the like.

The term “pharmaceutically acceptable” as used herein means suited for normal pharmaceutical applications, i.e. giving rise to no adverse events in patients etc.

The term “excipient” as used herein means the chemical compounds which are normally added to pharmaceutical compositions, e.g. buffers, tonicity agents, preservatives and the like.

The term “effective amount” as used herein means a dosage which is sufficient to be effective for the treatment of the patient compared with no treatment.

The term “pharmaceutical composition” as used herein means a product comprising an active compound or a salt thereof together with pharmaceutical excipients such as buffer, preservative, and optionally a tonicity modifier and/or a stabilizer. Thus a pharmaceutical composition is also known in the art as a pharmaceutical formulation.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

### DESCRIPTION OF THE INVENTION

In one aspect the present invention relates to a compound having the structure of the formula (I):
GLP-1 agonist-L-RR-protraction protein (I)
wherein
GLP-1 agonist is a polypeptide which is an agonist of the human GLP-1 receptor,
L is a linker connecting an amino acid side chain of said GLP-1 agonist or the C-terminal amino acid residue of said GLP-1 agonist with RR,
RR is the remains of a reactive residue that has formed a covalent bond with an amino acid residue of the protraction protein, and
protraction protein is a protein having a molar weight of at least 5 kDa, having a plasma half-life of at least 24 hours in human plasma, and said protraction protein has been synthesised by a non-mammalian organism or synthetically.

The compounds encompassed by formula (I) is illustrated by the following illustration

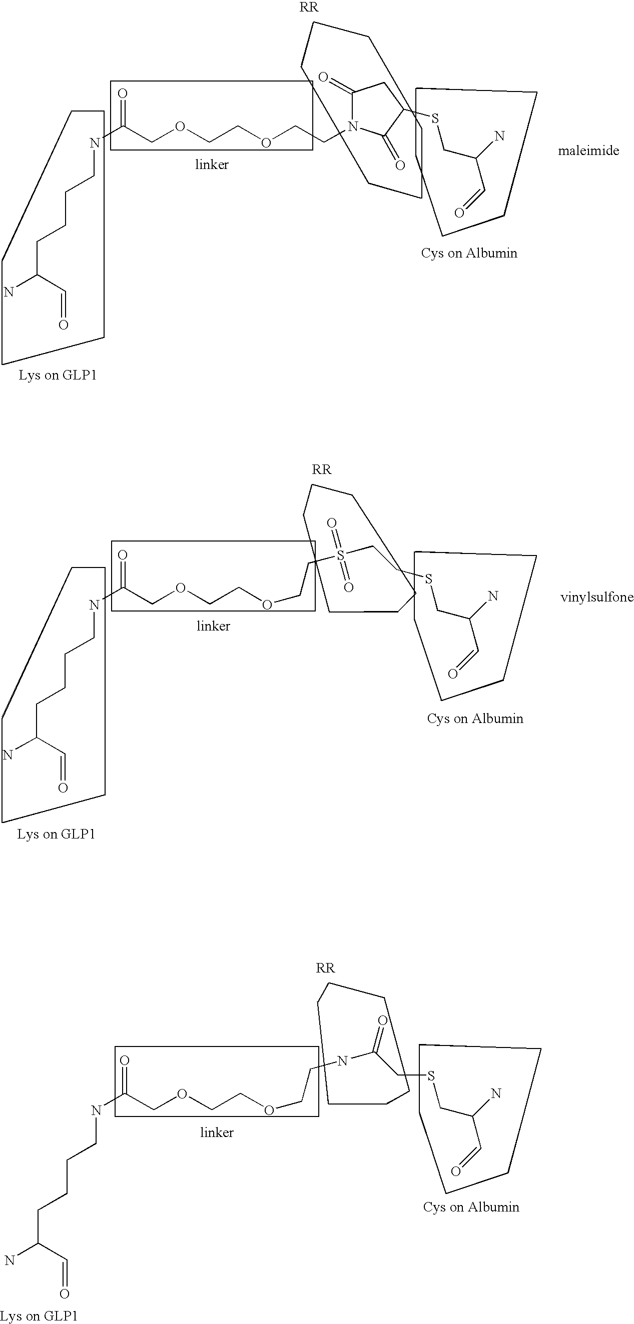

In one embodiment of the invention the protraction protein is recombinant human serum albumin (SEQ ID NO 1).

In another embodiment of the invention the protraction protein is a human serum albumin variant.

In another embodiment of the invention the human serum albumin variant has reduced binding affinities towards copper and nickel as compared to the corresponding binding affinities of human serum albumin towards copper and nickel.

In another embodiment of the invention the protraction protein is an N-terminal fragment of human serum albumin, or an analogue thereof.

In another embodiment of the invention the protraction protein is a human serum albumin variant comprising a modification of the Asp-Ala-His-Lys N-terminal sequence.

In another embodiment of the invention the protraction protein comprises at least one deletion among the three N-terminal amino acid residues Asp-Ala-His.

In another embodiment of the invention the protraction protein comprises an N-terminal extension, such as Glu⁻³,Ala⁻²Glu⁻¹,Phe⁰-HSA(1-585) or an N-terminal fragment thereof.

In another embodiment of the invention the human serum albumin (HSA) variant is selected from the group consisting of HSA(2-585), HSA(3-585), HSA(4-585), Asp-Ala-HSA(4-585), Xaa³-HSA(1-585) where Xaa³ is an amino acid residue which has substituted the His residue occupying position 3 in native HSA, and N-terminal fragments thereof.

A recombinant human serum albumin variant is commercially available from New Century Pharma under the name Albagen. Albagen is HSA(2-585) and is hypoallergenic due to the modified metal binding properties caused by the single N-terminal deletion.

In another embodiment of the invention the said protraction protein comprises an amino acid sequence of from 60-200 such as from 100 to 150 amino acid residues, and said amino acid sequence being identical to a fragment of SEQ ID NO 1 or a fragment of SEQ ID NO 1 with one or two amino acid substitutions and/or deletions.

In another embodiment of the invention the protraction protein is the Fc portion of an immunoglobulin, an analogue or a fragment thereof.

In another embodiment of the invention the GLP-1 agonist has at least 50% amino acid homology with either GLP-1(7-37) (SEQ ID NO 2) or Exendin-4(1-39) (SEQ ID NO 3).

In another embodiment of the invention the GLP-1 agonist has at least 80% amino acid homology with either GLP-1(7-37) (SEQ ID NO 2) or Exendin-4(1-39) (SEQ ID NO 3).

In another embodiment of the invention the GLP-1 agonist comprises the amino acid sequence of the formula (II):
Xaa₇-Xaa₈-Glu-Gly-Thr-Phe-Thr-Ser-Asp-Xaa₁₆-Ser-Xaa₁₈-Xaa₁₉-Xaa₂₀-Glu-Xaa₂₂-Xaa₂₃-Ala-
Xaa₂₅-Xaa₂₆-Xaa₂₇-Phe-lIe-Xaa₃₀-Trp-Leu-Xaa₃₃-Xaa₃₄-Xaa₃₅-Xaa₃₆-Xaa₃₇-Xaa₃₈-Xaa₃₉-Xaa₄₀
-Xaa₄₁-Xaa₄₂-Xaa₄₃-Xaa₄₄-Xaa₄₅-Xaa₄₆ Formula (II) (SEQ ID No: 4)
wherein
Xaa₇ is L-histidine, D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine;
Xaa₈ is Ala, D-Ala, Gly, Val, Leu, Ile, Lys, Aib, (1-aminocyclopropyl)carboxylic acid, (1-aminocyclobutyl)carboxylic acid, 1-aminocyclopentyl)carboxylic acid, (1-aminocyclohexyl)carboxylic acid, (1-aminocycloheptyl)carboxylic acid, or (1-aminocyclooctyl)carboxylic acid;
Xaa₁₆ is Val or Leu;
Xaa₁₈ is Ser, Lys or Arg;
Xaa₁₉ is Tyr or Gln;
Xaa₂₀ is Leu or Met;
Xaa₂₂ is Gly, Glu or Aib;
Xaa₂₃ is Gln, Glu, Lys or Arg;
Xaa₂₅ is Ala or Val;
Xaa₂₆ is Lys, Glu or Arg;
Xaa₂₇ is Glu or Leu;
Xaa₃₀ is Ala, Glu or Arg;
Xaa₃₃ is Val or Lys;
Xaa₃₄ is Lys, Glu, Asn or Arg;
Xaa₃₅ is Gly or Aib;
Xaa₃₆ is Arg, Gly or Lys;
Xaa₃₇ is Gly, Ala, Glu, Pro, Lys, amide or is absent;
Xaa₃₈ is Lys, Ser, amide or is absent.
Xaa₃₉ is Ser, Lys, amide or is absent;
Xaa₄₀ is Gly, amide or is absent;
Xaa₄₁ is Ala, amide or is absent;
Xaa₄₂ is Pro, amide or is absent;
Xaa₄₃ is Pro, amide or is absent;
Xaa₄₄ is Pro, amide or is absent;
Xaa₄₅ is Ser, amide or is absent;
Xaa₄₆ is amide or is absent;
provided that if Xaa₃₈, Xaa₃₉, Xaa₄₀, Xaa₄₁, Xaa₄₂, Xaa₄₃, Xaa₄₄, Xaa₄₅ or Xaa₄₆ is absent then each amino acid residue downstream is also absent.

In another embodiment of the invention the GLP-1 agonist comprises the amino acid sequence of formula (III):
Xaa₇-Xaa₈-Glu-Gly-Thr-Phe-Thr-Ser-Asp-Val-Ser-Xaa₁₈-Tyr-Leu-Glu-Xaa₂₂-Xaa₂₃-Ala-Ala-
Xaa₂₆-Glu-Phe-Ile-Xaa₃₀-Trp-Leu-Val-Xaa₃₄-Xaa₃₅-Xaa₃₆-Xaa₃₇-Xaa₃₈ Formula (III) (SEQ ID No: 5)
wherein
Xaa₇ is L-histidine, D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine;
Xaa₈ is Ala, D-Ala, Gly, Val, Leu, Ile, Lys, Aib, (1-aminocyclopropyl)carboxylic acid, (1-aminocyclobutyl)carboxylic acid, 1-aminocyclopentyl)carboxylic acid, or (1-aminocyclohexyl)carboxylic acid, (1-aminocycloheptyl)carboxylic acid, or (1-aminocyclooctyl)carboxylic acid;
Xaa₁₈ is Ser, Lys or Arg;
Xaa₂₂ is Gly, Glu or Aib;
Xaa₂₃ is Gln, Glu, Lys or Arg;
Xaa₂₆ is Lys, Glu or Arg;
Xaa₃₀ is Ala, Glu or Arg;
Xaa₃₄ is Lys, Glu or Arg;
Xaa₃₅ is Gly or Aib;
Xaa₃₆ is Arg or Lys;
Xaa₃₇ is Gly, Ala, Glu or Lys;
Xaa₃₈ is Lys, amide or is absent.

In another embodiment of the invention the said GLP-1 agonist is dipeptidyl aminopeptidase IV protected. In another embodiment of the invention the GLP-1 agonist is hydrolysed by DPP-IV at a rate lower than the rate of hydrolysis of GLP-1 (7-37) using the DPP-IV hydrolysis assay disclosed herein.

In another embodiment of the invention the GLP-1 agonist is a position 8 analogue, i.e. the alanine residue in position 8 relative to the GLP-1 (7-37) sequence (SEQ ID No: 2) has been substituted by another amino acid residue.

In another embodiment of the invention the GLP-1 agonist comprises an Aib residue in position 8 relative to the GLP-1(7-37) sequence (SEQ ID No:2).

In another embodiment of the invention the amino acid residue in position 7 of the GLP-1 peptide (the N-terminal) is selected from the group consisting of D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine and 4-pyridylalanine.

In another embodiment of the invention the GLP-1 agonist comprises no more than twelve amino acid residues which have been exchanged, added or deleted as compared to GLP-1(7-37) (SEQ ID No:2) or Exendin-4(1-39) (SEQ ID No:3).

In another embodiment of the invention the GLP-1 agonist comprises no more than six amino acid residues which have been exchanged, added or deleted as compared to GLP-1(7-37) (SEQ ID No:2) or Exendin-4(1-39) (SEQ ID No:3).

In another embodiment of the invention the GLP-1 agonist comprises no more than four amino acid residues which have been exchanged, added or deleted as compared to GLP-1(7-37) (SEQ ID No:2) or Exendin-4(1-39) (SEQ ID No:3).

In another embodiment of the invention the GLP-1 agonist comprises no more than 4 amino acid residues which are not encoded by the genetic code.

In another embodiment of the invention the GLP-1 agonist comprises no more than two amino acid residues which have been exchanged, added or deleted as compared to GLP-1(7-37) (SEQ ID No:2) or Exendin-4(1-39) (SEQ ID No:3).

In another embodiment of the invention the GLP-1 agonist is selected from the group consisting of [Arg³⁴]GLP-1 (7-37), [Arg^{26,34}]GLP-1 (7-37)Lys, [Lys³⁶Arg^{26,34}]GLP-1(7-36), [Aib^{8,22,35}]GLP-1 (7-37), [Aib^{8,35}]GLP-1(7-37), [Aib^{8,22}]GLP-1(7-37), [Aib^{8,22,35} Arg^{26,34}]GLP-1 (7-37) Lys, [Aib^{8,35} Arg^{26,34}]GLP-1 (7-37)Lys, [Aib^{8,22} Arg^{26,34}]GLP-1 (7-37)Lys, [Aib^{8,22,35} Arg^{26,34}]GLP-1 (7-37)Lys, [Aib^{8,35} Arg^{26,34}]GLP-1 (7-37)Lys, [Aib^{8,22,35} Arg²⁶]GLP-1(7-37) Lys, [Aib^{8,35} Arg²⁶]GLP-1(7-37) Lys, [Aib^{8,22} Arg²⁶]GLP-1 (7-37)Lys, [Aib^{8,22,35} Arg³⁴]GLP-1 (7-37) Lys, [Aib^{8,35}Arg³⁴]GLP-1(7-37)Lys, [Aib^{8,22}Arg³⁴]GLP-1 (7-37)Lys, [Aib^{8,22,35}Ala³⁷]GLP-1 (7-37)Lys, [Aib^{8,35}Ala³⁷]GLP-1 (7-37)Lys, [Aib^{8,22}Ala³⁷]GLP-1 (7-37)Lys, [Aib^{8,22,35} Lys³⁷]GLP-1(7-37), [Aib^{8,35}Lys³⁷]GLP-1 (7-37) and [Aib^{8,22}Lys³⁷]GLP-1 (7-37).

In another embodiment of the invention the GLP-1 agonist is Exendin-4(1-39) (SEQ ID No. 3).

In another embodiment of the invention the GLP-1 agonist is ZP-10, i.e. [Ser³⁸Lys³⁹]Exendin-4(1-39)LysLysLysLysLys-amide (SEQ ID No. 4).

In another embodiment of the invention the GLP-1 agonist is attached to the moiety: -L-RR-protraction protein via the side chain of the amino acid residue in position 23, 26, 34, 36 or 38 relative to the amino acid sequence SEQ ID No:2 (GLP-1(7-37)), (corresponding to position 17, 20, 28, 30 or 32 relative to amino acid sequence SEQ ID No:3 (Exendin-4(1-39)).

In another embodiment of the invention the GLP-1 agonist is attached to the moiety: -L-RR-protraction protein via the side chain of the C-terminal amino acid residue.

In another embodiment of the invention the GLP-1 agonist is attached to the moiety: -L-RR-protraction protein via the side chain of an amino acid residue selected from arginine, lysine, cysteine, glutamic acid, aspartic acid, histidine, serine, threonine and tyrosine.

In another embodiment of the invention the GLP-1 agonist is attached to the moiety: -L-RR-protraction protein via the side chain of a cysteine residue.

In another embodiment of the invention the linker L is selected from the group consisting of the bivalent connecting chemical groups

amides: —C(O)—NR—, where R is hydrogen or C₁₋₆-alkyl,

amine: —NR—, where R is hydrogen or C₁₋₆-alkyl,

thioethers: —S—, —S—(CH₂)₂—SO₂— or Error! Objects cannot be created from editing field codes.,

ethers: —O—,

urethanes: —N(R¹)—CO—N(R²)—, where R¹ and R² independently is hydrogen or C₁₋₆-alkyl,

carbamates: —O—CO—N(R)—, where R is hydrogen or C₁₋₆-alkyl,

hydrazines: Error! Objects cannot be created from editing field codes. where R is hydrogen or C₁₋₆-alkyl,

oximes: —O—N═C(—R)—, where R is hydrogen or C₁₋₆-alkyl,

oxazolidines or thiazolidines:

Error! Objects cannot be created from editing field codes., and

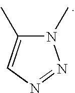

In another embodiment of the invention the compound of general formula (I) is selected from the group consisting of

GLP-1 agonist —C(═O)CH₂O(CH₂)₂O(CH₂)₂—RR-protraction protein,

GLP-1 agonist —C(═O)(CH₂)ₙ(OCH₂CH₂)ₘ—RR-protraction protein,

GLP-1 agonist —S(═O)₂(CH₂)ₙ(OCH₂CH₂)ₘ—RR-protraction protein,

GLP-1 agonist-CH₂(CH₂)ₙ(OCH₂CH₂)ₘ—RR-protraction protein,

GLP-1 agonist —C(═O)O(CH₂)ₙ(OCH₂CH₂)ₘ—RR-protraction protein,

wherein n is an integer in the range from 0 to 10, and m is an integer in the range from 0 to 100.

In another embodiment of the invention the compound of general formula (I) is selected from the group consisting of

GLP-1 agonist-L-NC(═O)CH₂— sulphur in cysteine residue in protraction protein,

GLP-1 agonist-L-S(═O)₂(CH₂)₂— sulphur in cysteine residue in protraction protein,

GLP-1 agonist-L-NC(═O)CH₂— sulphur in cysteine residue in protraction protein, and

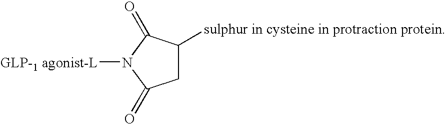

In another embodiment of the invention the compound of the general formula (I) is selected from the group consisting of S-gamma³⁴-(1-{2-[2-(2-([D-Ala⁸, Lys³⁷]-GLP-1-(7-37)amide -N^{ε37}-yl)acetyloxyethoxy)ethylcarbamoyl]ethyl}-2,5-dioxo-pyrrolidin-3-yl)Albagen 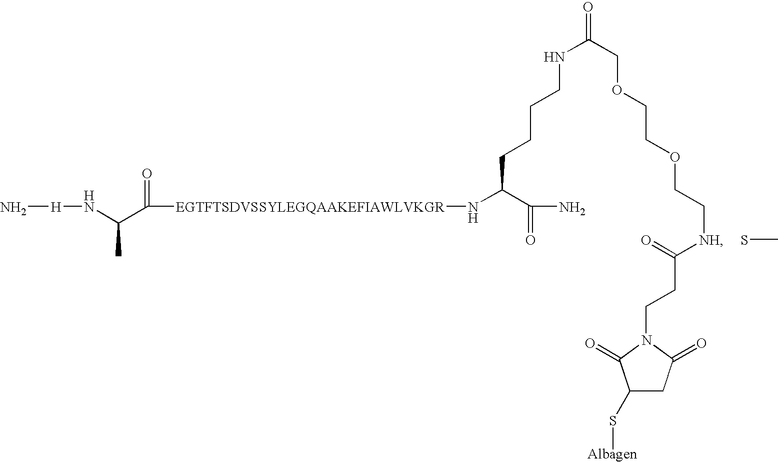
gamma³⁴-(1-{2-[2-(2-([Aib^{8,22,25}, Lys³⁷]-GLP-1-(7-37)amide-N^{ε37}-yl)acetyloxyethoxy)ethylcarbamoyl]ethyl}-2,5-dioxo-pyrrolidin-3-yl)Albagen 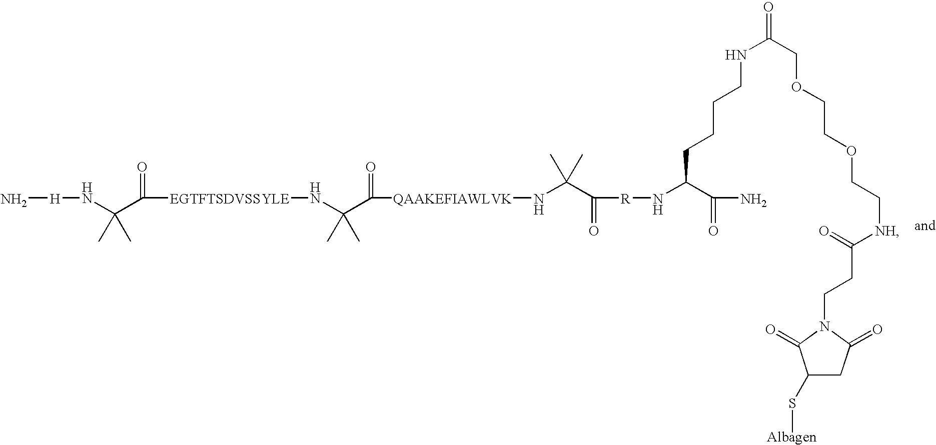
S-gamma³⁴-((1-{2-[2-(2-([Aib8,Arg26,34,Glu22,23,30]-GLP-1-(7-37))Lys amide-N^{ε}-yl)acetyloxyethoxy)ethylcarbamoyl]ethyl}-2,5-dioxo-pyrrolidin-3-yl)Albagen

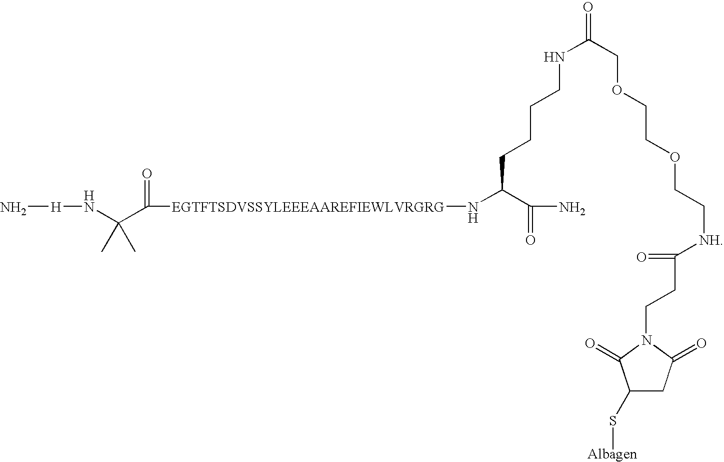

The compounds of the present invention can be produced by classical peptide synthesis, e.g. solid phase peptide synthesis using t-Boc or Fmoc chemistry or other well established techniques., see e.g. Green and Wuts, “Protecting Groups in Organic Synthesis”, John Wiley & Sons, 1999. These methods are preferred when the insulinotropic agent is a peptide comprising non-natural amino acid residues.

When the insulinotropic agent is a polypeptide comprising only amino acid residues encoded by the genetic code, the polypeptides can also be produced by a method which comprises culturing a host cell containing a DNA sequence encoding the polypeptide and capable of expressing the polypeptide in a suitable nutrient medium under conditions permitting the expression of the peptide, after which the resulting peptide is recovered from the culture and then derivatized to the compound of formula (I).

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The peptide produced by the cells may then be recovered from the culture medium by conventional procedures including separating the host cells from the medium by centrifugation or filtration. For extracellular products the proteinaceous components of the supernatant are isolated by filtration, column chromatography or precipitation, e.g. microfiltration, ultrafiltration, isoelectric precipitation, purification by a variety of chromatographic procedures, e.g. ion exchange chromatography, hydrophobic interaction chromatography, gel filtration chromatography, affinity chromatography, or the like, dependent on the type of polypeptide in question. For intracellular or periplasmic products the cells isolated from the culture medium are disintegrated or permeabilised and extracted to recover the product polypeptide or precursor thereof.

The DNA sequence encoding the therapeutic polypeptide may suitably be of genomic or cDNA origin, for instance obtained by preparing a genomic or cDNA library and screening for DNA sequences coding for all or part of the peptide by hybridisation using synthetic oligonucleotide probes in accordance with standard techniques (see, for example, Sambrook, J, Fritsch, E F and Maniatis, T, Molecular Cloning: A Laboratory Manual, Cold Spring Harbor Laboratory Press, New York, 1989). The DNA sequence encoding the polypeptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. The DNA sequence may also be prepared by polymerase chain reaction using specific primers, for instance as described in U.S. Pat. No. 4,683,202 or Saiki et al., Science 239 (1988), 487-491.

The DNA sequence may be inserted into any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the polypeptide is operably linked to additional segments required for transcription of the DNA, such as a promoter. The promoter may be any DNA sequence which shows transcriptional activity in the host cell of choice and may be derived from genes encoding proteins either homologous or heterologous to the host cell. Examples of suitable promoters for directing the transcription of the DNA encoding the peptide of the invention in a variety of host cells are well known in the art, cf. for instance Sambrook et al., supra.

The DNA sequence encoding the polypeptide may also, if necessary, be operably connected to a suitable terminator, polyadenylation signals, transcriptional enhancer sequences, and translational enhancer sequences. The recombinant vector of the invention may further comprise a DNA sequence enabling the vector to replicate in the host cell in question.

The vector may also comprise a selectable marker, e.g. a gene the product of which complements a defect in the host cell or one which confers resistance to a drug, e.g. ampicillin, kanamycin, tetracyclin, chloramphenicol, neomycin, hygromycin or methotrexate. For large scale manufacture the selectable marker preferably is not antibiotic resistance, e.g. antibiotic resistance genes in the vector are preferably excised when the vector is used for large scale manufacture. Methods for eliminating antibiotic resistance genes from vectors are known in the art, see e.g. U.S. Pat. No. 6,358,705 which is incorporated herein by reference.

To direct a parent peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

The procedures used to ligate the DNA sequences coding for the present peptide, the promoter and optionally the terminator and/or secretory signal sequence, respectively, and to insert them into suitable vectors containing the information necessary for replication, are well known to persons skilled in the art (cf., for instance, Sambrook et al., supra).

The host cell into which the DNA sequence or the recombinant vector is introduced may be any cell which is capable of producing the present peptide and includes bacteria, yeast, fungi and higher eukaryotic cells. Examples of suitable host cells well known and used in the art are, without limitation, E. coli, Saccharomyces cerevisiae, or mammalian BHK or CHO cell lines.

Pharmaceutical compositions containing a compound according to the present invention may be prepared by conventional techniques, e.g. as described in Remington's Pharmaceutical Sciences, 1985 or in Remington: The Science and Practice of Pharmacy, 19^{th} edition, 1995.

One object of the present invention is to provide a pharmaceutical formulation comprising a compound according to the present invention which is present in a concentration from about 0.1 mg/ml to about 25 mg/ml, and wherein said formulation has a pH from 2.0 to 10.0. The formulation may further comprise a buffer system, preservative(s), isotonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of a compound according to the present invention, and a buffer, wherein said compound is present in a concentration from 0.1 mg/ml or above, and wherein said formulation has a pH from about 2.0 to about 10.0.

In another embodiment of the invention the pH of the formulation is from about 7.0 to about 9.5. In another embodiment of the invention the pH of the formulation is from about 3.0 to about 7.0. In another embodiment of the invention the pH of the formulation is from about 5.0 to about 7.5. In another embodiment of the invention the pH of the formulation is from about 7.5 to about 9.0. In another embodiment of the invention the pH of the formulation is from about 7.5 to about 8.5. In another embodiment of the invention the pH of the formulation is from about 6.0 to about 7.5. In another embodiment of the invention the pH of the formulation is from about 6.0 to about 7.0.

In another embodiment of the invention the pH of the formulation is from about 3.0 to about 9.0, and said pH is at least 2.0 pH units from the isoelectric pH of compound of the present invention.

In a further embodiment of the invention the buffer is selected from the group consisting of sodium acetate, sodium carbonate, citrate, glycylglycine, histidine, glycine, lysine, arginin, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof.

In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di- or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used.

In one embodiment the sugar additive is sucrose. Sugar alcohol is defined as a C₄-C₈ hydrocarbon having at least one —OH group and includes, for example, mannitol, sorbitol, inositol, galacititol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabiliser. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or DL isomer) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include S-ethyl homocysteine and S-butyl homocysteine and suitable cystein analogues include S-methyl-L cystein. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphur containing amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D, or DL isomer) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabiliser selected from the group of high molecular weight polymers or low molecular compounds. In a further embodiment of the invention the stabilizer is selected from polyethylene glycol (e.g. PEG 3350), polyvinylalcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides,
sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lecitins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)- derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives- (e.g. sodium tauro-dihydrofusidates etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quarternary ammonium bases) (e.g. cetyl-trimethylammonium bromide, cetylpyridinium chloride), non-ionic surfactants (eg. dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a compound according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

In one aspect the present invention relates to a pharmaceutical composition comprising a compound according to Formula (I), and a pharmaceutically acceptable excipient.

In one embodiment the pharmaceutical composition is suited for pulmonary administration.

In another aspect the present invention relates to the use of a compound of formula (I) for the preparation of a pulmonary medicament.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of the compound, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles, Methods to produce controlled release systems useful for compositions of the current invention include, but are not limited to, crystallization, condensation, co-crystallization, precipitation, co-precipitation, emulsification, dispersion, high pressure homogenization, encapsulation, spray drying, microencapsulation, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound according to the present invention in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the compound according to the present invention is stable for more than 6 weeks of usage and for more than 3 years of storage.

In another embodiment of the invention the pharmaceutical formulation comprising the compound according to the present invention is stable for more than 4 weeks of usage and for more than 3 years of storage.

In a further embodiment of the invention the pharmaceutical formulation comprising the compound according to the present invention is stable for more than 4 weeks of usage and for more than two years of storage.

In an even further embodiment of the invention the pharmaceutical formulation comprising the compound is stable for more than 2 weeks of usage and for more than two years of storage.

In another aspect the present invention relates to the use of a compound according to the invention for the preparation of a medicament.

In one embodiment a compound according to the invention is used for the preparation of a medicament for the treatment or prevention of hyperglycemia, type 2 diabetes, impaired glucose tolerance, type 1 diabetes, obesity, hypertension, syndrome X, dyslipidemia, cognitive disorders, atheroschlerosis, myocardial infarction, coronary heart disease and other cardiovascular disorders, stroke, inflammatory bowel syndrome, dyspepsia and gastric ulcers.

In another embodiment a compound according to the invention is used for the preparation of a medicament for delaying or preventing disease progression in type 2 diabetes.

In another embodiment a compound according to the invention is used for the preparation of a medicament for decreasing food intake, decreasing β-cell apoptosis, increasing β-cell function and β-cell mass, and/or for restoring glucose sensitivity to β-cells.

The treatment with a compound according to the present invention may also be combined with combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists.

It should be understood that any suitable combination of the compounds according to the invention with one or more of the above-mentioned compounds and optionally one or more further pharmacologically active substances are considered to be within the scope of the present invention.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

In another aspect the present invention relates to a pharmaceutical composition comprising a compound according to general formula (I), and a pharmaceutically acceptable preservative.

In one embodiment of the invention the pharmaceutical composition comprises a compound according to the general formula (I) and a pharmaceutically acceptable stabilizer.

In another embodiment of the invention the pharmaceutical composition is suited for parenteral administration.

In another aspect the present invention relates to the use of a compound according to the general formula (I) for the preparation of a medicament.

### EXAMPLES

General Procedure (A)

General Synthetic Methods

The peptide was synthesized on Fmoc protected Rink amide resin (Novabiochem) or chlorotrityl resin using Fmoc strategy on an Applied Biosystems 433A peptide synthesizer in 0.25 mmol scale using the manufacturer supplied FastMoc UV protocols which employ HBTU (2-(1H-Benzotriazol-1-yl-)-1,1,3,3 tetramethyluronium hexafluorophosphate) mediated couplings in N-methylpyrrolidone (N-methyl pyrrolidone) and UV monitoring of the deprotection of the Fmoc protection group. The protected amino acid derivatives used were standard Fmoc-amino acids (Anaspec) supplied in preweighed cartridges suitable for the ABI433A synthesizer with the exception of unnatural aminoacids such as Fmoc-Aib-OH (Fmoc-aminoisobutyric acid).

The attachment of sidechains and linkers to specific lysine residues on the crude resin bound protected peptide was carried out in a specific position by incorporation of Fmoc-Lys(Dde)-OH during automated synthesis.

Procedure for removal of Dde-protection. The resin (0.25 mmol) was placed in a manual shaker/filtration apparatus and treated with 2% hydrazine in N-methyl pyrrolidone (20 ml, 2×12 min) to remove the DDE group and wash with N-methyl pyrrolidone (4×20 ml).

Procedure for Attachment of Sidechains to Lysine Residues.

The amino acid (4 molar equivalents relative to resin) was dissolved in N-methyl pyrrolidone/methylene chloride (1:1, 20 ml). Hydroxybenzotriazole (HOBt) (4 molar equivalents relative to resin) and diisopropylcarbodiimide (4 molar equivalents relative to resin) was added and the solution was stirred for 15 min. The solution was added to the resin and diisopropyethylamine (4 molar equivalents relative to resin) was added. The resin was shaken 24 hours at room temperature. The resin was washed with N-methylpyrrolidone (2×20 ml), N-methylpyrrolidone/Methylene chloride (1:1) (2×20 ml) and methylene chloride (2×20 ml).

Procedure for removal of Dde-protection: The resin (0.25 mmol) was placed in a filter flask in a manual shaking apparatus and treated with N-methyl pyrrolidone/methylene chloride (1:1) (2×20 ml) and with N-methylpyrrolidone (1×20 ml), a solution of 20% piperidine in N-methylpyrrolidone (3×20 ml, 10 min each). The resin was washed with N-methylpyrrolidone (2×20 ml), N-methylpyrrolidone/Methylene chloride (1:1) (2×20 ml) and methylene chloride (2×20 ml).

Procedure for Cleaving the Peptide Off the Resin:

The peptide was cleaved from the resin by stirring for 180 min at room temperature with a mixture of trifluoroacetic acid, water and triisopropylsilane (95:2.5:2.5). The cleavage mixture was filtered and the filtrate was concentrated to an oil by a stream of nitrogen. The crude peptide was precipitated from this oil with 45 ml diethyl ether and washed 3 times with 45 ml diethyl ether.

Purification: The crude peptide was purified by semipreparative HPLC on a 20 mm×250 mm column packed with 7μ C-18 silica. Depending on the peptide two one or two purification systems were used.

TFA: After drying the crude peptide was dissolved in 5 ml 50% acetic acid H₂O and diluted to 20 ml with H₂O and injected on the column which then was eluted with a gradient of 40-60% CH₃CN in 0.1% TFA 10 ml/min during 50 min at 40° C. The peptide containing fractions were collected. The purified peptide was lyophilized after dilution of the eluate with water.

Ammonium sulphate: The column was equilibrated with 40% CH₃CN in 0.05M (NH₄)₂SO₄, which was adjusted to pH 2.5 with concentrated H₂SO₄. After drying the crude peptide was dissolved in 5 ml 50% acetic acid H₂O and diluted to 20 ml with H₂O and injected on the column which then was eluted with a gradient of 40%-60% CH₃CN in 0.05M (NH₄)₂SO₄, pH 2.5 at 10 ml/min during 50 min at 40° C. The peptide containing fractions were collected and diluted with 3 volumes of H₂O and passed through a Sep-Pak® C18 cartridge (Waters part. #:51910) which has been equilibrated with 0.1% TFA. It was then eluted with 70% CH₃CN containing 0.1% TFA and the purified peptide was isolated by lyophilisation after dilution of the eluate with water.

The final product obtained was characterised by analytical RP-HPLC (retention time) and by LCMS

The RP-HPLC analysis was performed using UV detection at 214 nm and a Vydac 218TP54 4.6 mm×250 mm 5μ C-18 silica column (The Separations Group, Hesperia, USA) which was eluted at 1 ml/min at 42° C. Two different elution conditions were used:
A1: Equilibration of the column with in a buffer consisting of 0.1M (NH₄)₂SO₄, which was adjusted to pH 2.5 with concentrated H₂SO₄ and elution by a gradient of 0% to 60% CH₃CN in the same buffer during 50 min.
B1: Equilibration of the column with 0.1% TFA/H₂O and elution by a gradient of 0% CH₃CN/0.1% TFA/H₂O to 60% CH₃CN/0.1% TFA/H₂O during 50 min.
B6: Equilibration of the column with 0.1% TFA/H₂O and elution by a gradient of 0% CH₃CN/0.1% TFA/H₂O to 90% CH₃CN/0.1% TFA/H₂O during 50 min.

LCMS was performed on a setup consisting of Hewlett Packard series 1100 G1312A Bin Pump, Hewlett Packard series 1100 Column compartment, Hewlett Packard series 1100 G1315A DAD diode array detector, Hewlett Packard series 1100 MSD and Sedere 75 Evaporative Light Scattering detector controlled by HP Chemstation software. The HPLC pump is connected to two eluent reservoirs containing:

A: 10 mM NH₄OH in water

B: 10 mM NH₄OH in 90% acetonitrile

The analysis was performed at 23° C. by injecting an appropriate volume of the sample (preferably 20 μl) onto the column which is eluted with a gradient of A and B.

The HPLC conditions, detector settings and mass spectrometer settings used are giving in the following table.

Column Waters Xterra MS C-1 8×3 mm id 5 μm

Gradient 5%-100% acetonitrile linear during 6.5 min at 1.5 ml/min

Detection 210 nm (analogue output from DAD)

ELS (analogue output from ELS)

MS ionisation mode API-ES. Scan 100-1000 amu step 0.1 amu

Matrix Assisted Laser Desorption Ionization Mass spectrometric analysis (MALDI-MS) was performed on a Voyager RP MALDI-TOF instrument (Perseptive Biosystems Inc., Framingham, Mass.) equipped with a nitrogen laser (337 nm). The instrument was operated in linear mode with delayed extraction, and the accelerating voltage in the ion source was 25 kV. Sample preparation was done as follows: 1 μl sample-solution (0.5-1.0 mg/ml) was mixed with 10 μl matrix-solution (Sinapinic acid dissolved in a 5:4:1 mixture of acetonitrile:water:3% TFA) and 1 μl was deposited on the sample plate and allowed to dry.

Only external calibration was performed as the normal peptide standards used are in the low molecular weight range and insufficient to assure proper determination of masses in the range of serum albumin (>60 KDa). As a result the absolute mass values determined are only within 0.2% accuracy.

### Example 1

### S-gamma³⁴-(1-{2-[2-(2-([D-Ala⁸, Lys³⁷]-GLP-1-(7-37)amide-N^{ε37}-yl)acetyloxyethoxy)ethylcarbamoyl]ethyl}-2,5-dioxo-pyrrolidin-3-yl)Albagen

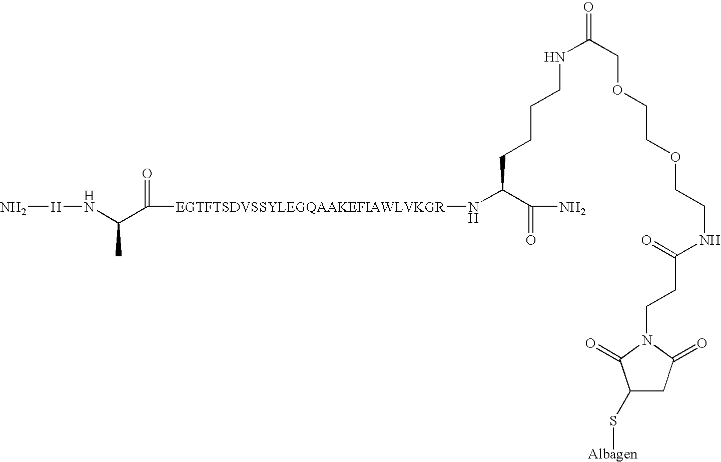

A resin (Rink amide, 0.68 mmol/g Novabiochem 0.25 mmole) was used to produce the primary sequence on an ABI433A machine according to manufacturers guidelines. All protecting groups were acid labile with the exception of the residue used in position 37 (FmocLys(ivDde)-OH, Novabiochem) allowing specific deprotection of this lysine rather than any other lysine.

Procedure

The resin (0.25 mmole) was placed in a manual shaker/filtration apparatus and treated with 2% hydrazine in N-methylpyrrolidone in (2×12 min. 2×20 ml) to remove the Dde group. The resin was washed with N-methylpyrrolidone (4×20 ml). Fmoc-8-amino-3,6-dioxaoctanoic acid (Neosystem FA03202) (4 molar equivalents relative to resin) was dissolved in N-methylpyrrolidone/methylene chloride (1:1, 20 ml). Hydroxybenzotriazole (HOBt) (4 molar equivalents relative to resin) and diisopropylcarbodiimide (4 molar equivalents relative to resin) was added and the solution was stirred for 15 min. The solution was added to the resin and diisopropylethylamine (4 molar equivalents relative to resin) was added. The resin was shaken 24 hours at room temperature. The resin was washed with N-methylpyrrolidone (4×20 ml). A solution of 20% piperidine in N-methylpyrrolidone (3×20 ml, 10 min each) was added to the resin while shaking. The resin was washed with N-methylpyrrolidone (4×20 ml). 3-maleimido propionic acid (4 molar equivalents relative to resin) was dissolved in N-methylpyrrolidone/methylene chloride (1:1, 20 ml). Hydroxybenzotriazole hydrate (HOBt; H₂O) (4 molar equivalents relative to resin) and diisopropylcarbodiimide (4 molar equivalents relative to resin) was added and the solution was stirred for 15 min. The solution was added to the resin and diisopropylethylamine (4 molar equivalents relative to resin) was added. The resin was shaken 24 hours at room temperature. The resin was washed with N-methylpyrrolidone (2×20 ml), N-methylpyrrolidone/methylene chloride (1:1) (2×20 ml) and methylene chloride (2×20 ml). The peptide was cleaved from the resin by stirring for 180 min at room temperature with a mixture of trifluoroacetic acid, water and triisopropylsilane (95:2.5:2.5). The cleavage mixture was filtered and the filtrate was concentrated to an oil by a stream of nitrogen. The crude peptide was precipitated from this oil with 45 ml diethyl ether and washed 3 times with 45 ml diethyl ether. The crude peptide was purified by semipreparative HPLC on a 20 mm×250 mm column packed with 7μ C-18 silica. The crude peptide was dissolved in 5 ml 50% acetic acid in water and diluted to 20 ml with H₂O and injected on the column which then was eluted with a gradient of 40-60% (CH₃CN in water with 0.1% TFA) 10 ml/min during 50 min at 40° C. The peptide containing fractions were collected. The purified peptide was lyophilized after dilution of the eluate with water affording N^{ε37}-(2-(2-(3-(maleimido)propionylamino)ethoxy)ethoxy)acetyl)[D-Ala⁸,Lys³⁷] GLP-1 (7-37)amide.

HPLC: (method B6): RT=36.8 min

HPLC: (method A1): RT=35.1 min

LCMS: m/z=931.4 (M+H)⁴⁺, 1241.5 (M+H)³⁺. Calculated (M+H)⁺=3722.1 Freeze dried N^{ε37}-(2-(2-(3-(maleimido)propionylamino)ethoxy)ethoxy)acetyl)[D-Ala⁸,Lys³⁷] GLP-1 (7-37)amide was dissolved in 10 μl 10% acetic acid and 800 μl 40 mg/ml Albagen (New Century Pharma) in 50 mM NaPi pH 7.0+4% hydroxypropyl-β-cyclodextrin (HP-β-CD) was added and stirred for 2 hours at ambient temperature. Subsequently, solid ammonium sulphate was added slowly to a final concentration of 2M. The conjugate was purified on a Resource™ HIC ISO with a total volume of 1 ml. Flow rate was kept at 1 ml/min. Buffer: 50 mM NaPi pH 7.0+4% HP-β-CD. A gradient from 2 M to 0 M ammonium sulphate over 20 column volumes was used to separate Albagen from the conjugate. The chromatogram showed two eluting peaks. The first peak was due to Albagen, the second peak contained the conjugate. The conjugate was concentrated and separated from non-reacted analogue on a centriprep™ device with a MW cut-off at 30,000 Da. The overall yield was 25-35%. The site of conjugation was determined to be Cys-34 by peptide mapping.

The mass found by MALDI is 70023 Da

Theoretical molecular weight of conjugate is 70046 Da.

### Example 2

### S-gamma³⁴-(1-{2-[2-(2-([Aib^{8,22,25}, Lys³⁷]-GLP-1-(7-37)amide-N^{ε37}-yl)acetyloxyethoxy)ethylcarbamoyl]ethyl}-2,5-dioxo-pyrrolidin-3-yl)Albagen

This compound was prepared as in example 1.

Data for GLP1 precursor

HPLC: (method B1): RT=38.5 min

HPLC: (method A1): RT=36.9 min

LCMS: m/z=949.0 (M+H)⁴⁺, 1264.9 (M+H)³⁺. Calculated (M+H)⁺=3792.2 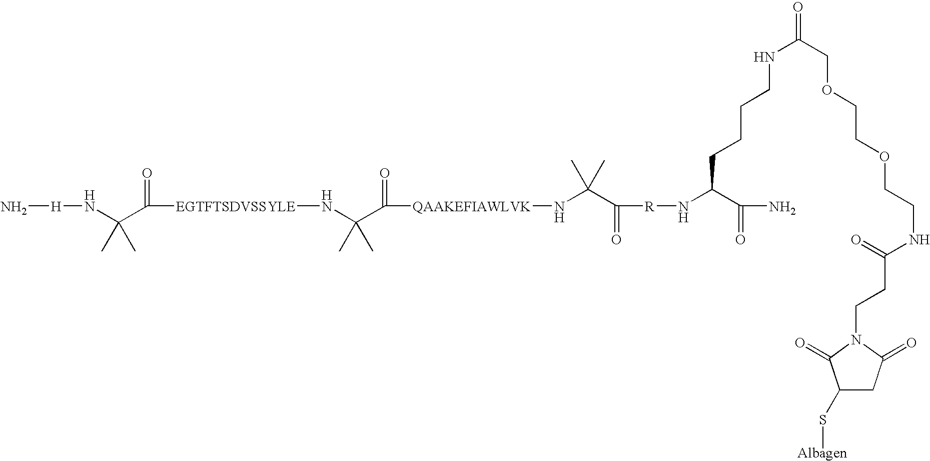
The mass found by MALDI is 70102 Da.
Theoretical molecular weight of conjugate is 70116 Da.

### Example 3

### S-gamma³⁴-((1-{2-[2-(2-([Aib8,Arg26,34,Glu22,23,30]-GLP-1-(7-37))Lys amide-N^{ε}-yl)acetyloxyethoxy)ethylcarbamoyl]ethyl}-2,5-dioxo-pyrrolidin-3-yl)Albagen

This compound was prepared as in example 1.

Data for GLP1-precursor

HPLC: (method B1): RT=36.5 min

HPLC: (method A1): RT=34.9 min

LCMS: m/z=(M+H)³⁺=1327.8 Calculated (M+H)⁺=3980.3 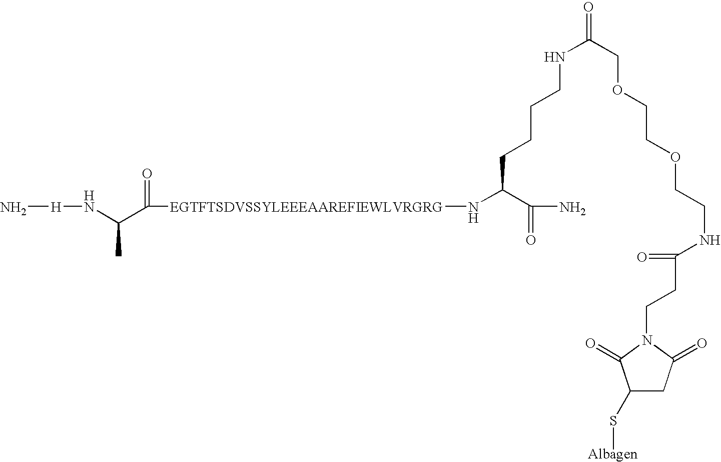
The mass found by MALDI is 70208 Da.
Theoretical molecular weight of conjugate is 70304 Da.

### Example 4

Other compounds which are synthesised according to the method described in example 1 are:

S-γ³⁴-(1-{2-[2-(2-([Lys³²]-exendin-(1-39)amide-N-ε³²-yl)acetyloxyethoxy)ethylcarbamoyl]ethyl}-2,5-dioxo-pyrrolidin-3-yl) Albumin. (wherein Albumin is recombinant Albagen from New Century Pharma, i.e. recombinant HSA(2-585)).
S-γ³⁴-(1-{2-[2-(2-([Lys²⁰]-exendin-(1-39)amide-N-ε²⁰-yl)acetyloxyethoxy)ethylcarbamoyl]ethyl}-2,5-dioxo-pyrrolidin-3-yl) Albumin. (wherein Albumin is recombinant Albagen from New Century Pharma, i.e. recombinant HSA(2-585)).
S-γ³⁴-(1-{2-[2-(2-([Arg², Lys²⁷]-exendin-(1-39)amide-N-ε²⁷-yl)acetyloxyethoxy)ethylcarbamoyl]ethyl}-2,5-dioxo-pyrrolidin-3-yl) Albumin. (Albumin is recombinant Albagen from New Century Pharma, i.e. recombinant HSA(2-585)).
S-γ³⁴-(1-{2-[2-(2-([Arg^{12,27}, Lys³²]-exendin-(1-39)amide-N-ε³²-yl)acetyloxyethoxy)ethylcarbamoyl]ethyl}-2,5-dioxo-pyrrolidin-3-yl) Albumin. (Albumin is recombinant Albagen from New Century Pharma, i.e. recombinant HSA(2-585)).

1. A compound of formula (I):
GLP-1 agonist-L-RR-protraction protein (I)
wherein
GLP-1 agonist is a polypeptide which is an agonist of the human GLP-1 receptor,
L is a linker connecting an amino acid side chain of said GLP-1 agonist or the C-terminal amino acid residue of said GLP-1 agonist with RR,
RR is the remains of a reactive residue that has formed a covalent bond with an amino acid residue of the protraction protein, and
protraction protein is a protein having a molar weight of at least 5 kDa, having a plasma half-life of at least 24 hours in human plasma, and said protraction protein has been synthesised by a non-mammalian organism or synthetically.

2. The compound according to claim 1, wherein said protraction protein is recombinant human serum albumin (SEQ ID NO 1).

3. The compound according to claim 1, wherein said protraction protein is a human serum albumin variant.

4. The compound according to claim 3, wherein said human serum albumin variant has reduced binding affinities towards copper and nickel as compared to the corresponding binding affinities of human serum albumin towards copper and nickel.

5. The compound according to claim 3, wherein said protraction protein is an N-terminal fragment of human serum albumin, or an analogue thereof.

6. The compound according to claim 3, wherein said protraction protein is a human serum albumin variant comprising a modification of the Asp-Ala-His-Lys N-terminal sequence.

7. The compound according to claim 6, wherein said protraction protein comprises at least one deletion among the three N-terminal amino acid residues Asp-Ala-His.

8. The compound according to claim 6, wherein said protraction protein comprises an N-terminal extension, such as Glu⁻³,Ala⁻²Glu⁻¹,Phe⁰-HSA(1-585) or an N-terminal fragment thereof.

9. The compound according to claim 6, wherein said human serum albumin (HSA) variant is selected from the group consisting of HSA(2-585), HSA(3-585), HSA(4-585), Asp-Ala-HSA(4-585), Xaa³-HSA(1-585) where Xaa³ is an amino acid residue which has substituted the His residue occupying position 3 in native HSA, and N-terminal fragments thereof.

10. The compound according to claim 1, wherein said protraction protein comprises an amino acid sequence of from 60-200 amino acid residues, said amino acid sequence being identical to a fragment of SEQ ID NO 1 or a fragment of SEQ ID NO 1 with one or two amino acid substitutions and/or deletions.

11. The compound according to claim 1, wherein said protraction protein is the Fc portion of an immunoglobulin, an analogue or a fragment thereof.

12. The compound according to claim 1, wherein said GLP-1 agonist has at least 50% amino acid homology with either GLP-1(7-37) (SEQ ID NO 2) or Exendin-4(1-39) (SEQ ID NO 3).

13. The compound according to claim 12, wherein said GLP-1 agonist has at least 80% amino acid homology with either GLP-1 (7-37) (SEQ ID NO 2) or Exendin-4(1-39) (SEQ ID NO 3).

14. The compound according to claim 1, wherein said GLP-1 agonist comprises the amino acid sequence of formula (II):
Xaa₇-Xaa₈-Glu-Gly-Thr-Phe-Thr-Ser-Asp-Xaa₁₆-Ser-Xaa₁₈-Xaa₁₉-Xaa₂₀-Glu-Xaa₂₂-Xaa₂₃-Ala-
Xaa₂₅-Xaa₂₆-Xaa₂₇-Phe-Ile-Xaa₃₀-Trp-Leu-Xaa₃₃-Xaa₃₄-Xaa₃₅-Xaa₃₆-Xaa₃₇-Xaa₃₈-Xaa₃₉-Xaa₄₀-
Xaa₄₁-Xaa₄₂-Xaa₄₃-Xaa₄₄-Xaa₄₅-Xaa₄₆ Formula (II) (SEQ ID No: 4)
wherein
Xaa₇ is L-histidine, D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine;
Xaa₈ is Ala, D-Ala, Gly, Val, Leu, Ile, Lys, Aib, (1-aminocyclopropyl)carboxylic acid, (1-aminocyclobutyl)carboxylic acid, 1-aminocyclopentyl)carboxylic acid, (1-aminocyclohexyl)carboxylic acid, (1-aminocycloheptyl)carboxylic acid, or (1-aminocyclooctyl)carboxylic acid;
Xaa₁₆ is Val or Leu;
Xaa₁₈ is Ser, Lys or Arg;
Xaa₁₉ is Tyr or Gln;
Xaa₂₀ is Leu or Met;
Xaa₂₂ is Gly, Glu or Aib;
Xaa₂₃ is Gln, Glu, Lys or Arg;
Xaa₂₅ is Ala or Val;
Xaa₂₆ is Lys, Glu or Arg;
Xaa₂₇ is Glu or Leu;
Xaa₃₀ is Ala, Glu or Arg;
Xaa₃₃ is Val or Lys;
Xaa₃₄ is Lys, Glu, Asn or Arg;
Xaa₃₅ is Gly or Aib;
Xaa₃₆ is Arg, Gly or Lys;
Xaa₃₇ is Gly, Ala, Glu, Pro, Lys, amide or is absent;
Xaa₃₈ is Lys, Ser, amide or is absent.
Xaa₃₉ is Ser, Lys, amide or is absent;
Xaa₄₀ is Gly, amide or is absent;
Xaa₄₁ is Ala, amide or is absent;
Xaa₄₂ is Pro, amide or is absent;
Xaa₄₃ is Pro, amide or is absent;
Xaa₄₄ is Pro, amide or is absent;
Xaa₄₅ is Ser, amide or is absent;
Xaa₄₆ is amide or is absent;
provided that if Xaa₃₈, Xaa₃₉, Xaa₄₀, Xaa₄₁, Xaa₄₂, Xaa₄₃, Xaa₄₄, Xaa₄₅ or Xaa₄₆ is absent then each amino acid residue downstream is also absent.

15. The compound according to claim 14, wherein said GLP-1 agonist comprises the amino acid sequence of formula (III):
Xaa₇-Xaa₈-Glu-Gly-Thr-Phe-Thr-Ser-Asp-Val-Ser-Xaa₁₈-Tyr-Leu-Glu-Xaa₂₂-Xaa₂₃-Ala-Ala-
Xaa₂₆-Glu-Phe-Ile-Xaa₃₀-Trp-Leu-Val-Xaa₃₄-Xaa₃₅-Xaa₃₆-Xaa₃₇-Xaa₃₈ Formula (III) (SEQ ID No: 5)
wherein
Xaa₇ is L-histidine, D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine;
Xaa₈ is Ala, D-Ala, Gly, Val, Leu, Ile, Lys, Aib, (1-aminocyclopropyl)carboxylic acid, (1-aminocyclobutyl)carboxylic acid, 1-aminocyclopentyl)carboxylic acid, or (1-aminocyclohexyl)carboxylic acid, (1-aminocycloheptyl)carboxylic acid, or (1-aminocyclooctyl)carboxylic acid;
Xaa₁₈ is Ser, Lys or Arg;
Xaa₂₂ is Gly, Glu or Aib;
Xaa₂₃ is Gln, Glu, Lys or Arg;
Xaa₂₆ is Lys, Glu or Arg;
Xaa₃₀ is Ala, Glu or Arg;
Xaa₃₄ is Lys, Glu or Arg;
Xaa₃₅ is Gly or Aib;
Xaa₃₆ is Arg or Lys;
Xaa₃₇ is Gly, Ala, Glu or Lys;
Xaa₃₈ is Lys, amide or is absent.

16. The compound according to claim 1, wherein said GLP-1 agonist is dipeptidyl aminopeptidase IV protected.

17. The compound according to claim 16, wherein said GLP-1 agonist is a position 8 analogue.

18. The compound according to claim 17, wherein said GLP-1 agonist comprises an Aib residue in position 8 relative to the GLP-1(7-37) sequence (SEQ ID No:2).

19. The compound according to claim 1, wherein the amino acid residue in position 7 of said GLP-1 peptide (the N-terminal) is selected from the group consisting of D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine and 4-pyridylalanine.

20. The compound according to claim 1, wherein said GLP-1 agonist comprises no more than twelve amino acid residues which have been exchanged, added or deleted as compared to GLP-1(7-37) (SEQ ID No:2) or Exendin-4(1-39) (SEQ ID No:3).

21. The compound according to claim 1, wherein said GLP-1 agonist comprises no more than six amino acid residues which have been exchanged, added or deleted as compared to GLP-1(7-37) (SEQ ID No:2) or Exendin-4(1-39) (SEQ ID No:3).

22. The compound according to claim 1, wherein said GLP-1 agonist comprises no more than four amino acid residues which have been exchanged, added or deleted as compared to GLP-1(7-37) (SEQ ID No:2) or Exendin-4(1-39) (SEQ ID No:3).

23. The compound according to claim 1, wherein said GLP-1 agonist comprises no more than 4 amino acid residues which are not encoded by the genetic code.

24. The compound according to claim 1, wherein said GLP-1 agonist comprises no more than two amino acid residues which have been exchanged, added or deleted as compared to GLP-1(7-37) (SEQ ID No:2) or Exendin-4(1-39) (SEQ ID No:3).

25. The compound according to claim 1, wherein said GLP-1 agonist is selected from the group consisting of [Arg³⁴]GLP-1(7-37), [Arg^{26,34}]GLP-1(7-37)Lys, [Lys³⁶Arg^{26,34}]GLP-1(7-36),
[Aib^{8,22,35}]GLP-1(7-37),
[Aib^{8,35}]GLP-1(7-37), [Aib^{8,22}]GLP-1(7-37), [Aib^{8,22,35} Arg^{26,34}]GLP-1 (7-37)Lys,
[Aib^{8,35} Arg^{26,34}]GLP-1 (7-37)Lys, [Aib^{8,22} Arg^{26,34}]GLP-1 (7-37)Lys,
[Aib^{8,22,35} Arg^{26,34}]GLP-1 (7-37)Lys, [Aib^{8,35} Arg^{26,34}]GLP-1 (7-37)Lys,
[Aib^{8,22,35} Arg²⁶]GLP-1 (7-37)Lys, [Aib^{8,35} Arg²⁶]GLP-1 (7-37)Lys, [Aib^{8,22} Arg²⁶]GLP-1 (7-37) Lys,
[Aib^{8,22,35} Arg³⁴]GLP-1(7-37)Lys, [Aib^{8,35}Arg³⁴]GLP-1(7-37)Lys, [Aib^{8,22}Arg³⁴]GLP-1 (7-37)Lys,
[Aib^{8,22,35}Ala³⁷]GLP-1 (7-37)Lys, [Aib^{8,35}Ala³⁷]GLP-1 (7-37)Lys, [Aib^{8,22}Ala³⁷]GLP-1 (7-37)Lys,
[Aib^{8,22,35} Lys³⁷]GLP-1(7-37), [Aib^{8,35}Lys³⁷]GLP-1 (7-37) and [Aib^{8,22}Lys³⁷]GLP-1 (7-37).

26. The compound according to claim 1, wherein said GLP-1 agonist is Exendin-4(1-39) (SEQ ID No. 3).

27. The compound according to claim 1, wherein said GLP-1 agonist is ZP-10, i.e. [Ser³⁸Lys³⁹]Exendin-4(1-39)LysLysLysLysLys-amide (SEQ ID No. 4).

28. The compound according to claim 1, wherein said GLP-1 agonist is attached to the moiety:
-L-RR-protraction protein
via the side chain of the amino acid residue in position 23, 26, 34, 36 or 38 relative to the amino acid sequence SEQ ID No:2 (GLP-1 (7-37)), (corresponding to position 17, 20, 28, 30 or 32 relative to amino acid sequence SEQ ID No:3 (Exendin-4(1-39)).

29. The compound according to claim 1, wherein said GLP-1 agonist is attached to the moiety:
-L-RR-protraction protein
via the side chain of the C-terminal amino acid residue.

30. The compound according to claim 1, wherein said GLP-1 agonist is attached to the moiety:
-L-RR-protraction protein
via the side chain of an amino acid residue selected from arginine, lysine, cysteine, glutamic acid, aspartic acid, histidine, serine, threonine and tyrosine.

31. The compound according to claim 1, wherein said GLP-1 agonist is attached to the moiety:
-L-RR-protraction protein
via the side chain of a cysteine residue.

32. The compound according to claim 1, wherein said linker L is selected from the group consisting of the bivalent connecting chemical groups
amides: —C(O)—NR—, where R is hydrogen or C₁₋₆-alkyl,
amine: —NR—, where R is hydrogen or C₁₋₆-alkyl, 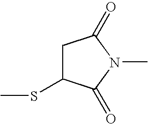
thioethers: —S—, —S—(CH₂)₂—SO₂— or
ethers: —O—,
urethanes: —N(R¹)CO—N(R²)—, where R¹ and R² independently is hydrogen or C₁₋₆alkyl,
carbamates: —O—CO—N(R), where R is hydrogen or C₁₋₆-alkyl,
hydrazines: 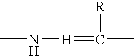
where R is hydrogen or C₁₋₆alkyl,
oximes: —O—N═C(—R)—, where R is hydrogen or C₁₋₆-alkyl,
oxazolidines or thiazolidines:

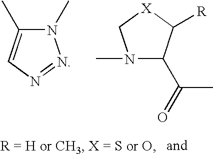

33. The compound according to claim 1, which is selected from the group consisting of
GLP-1 agonist —C(═O)CH₂O(CH₂)₂O(CH₂)₂—RR-protraction protein,
GLP-1 agonist-C(═O)(CH₂)ₙ(OCH₂CH₂)ₘ—RR-protraction protein,
GLP-1 agonist —S(═O)₂(CH₂)ₙ(OCH₂CH₂)ₘ—RR-protraction protein,
GLP-1 agonist —CH₂(CH₂)ₙ(OCH₂CH₂)ₘ—RR-protraction protein,
GLP-1 agonist —C(═O)O(CH₂)ₙ(OCH₂CH₂)ₘ—RR-protraction protein,
wherein n is an integer in the range from 0 to 10, and m is an integer in the range from 0 to 100.

34. The compound according to claim 1, which is selected from the group consisting of
GLP-1 agonist-L-NC(═O)CH₂— sulphur in cysteine residue in protraction protein,
GLP-1 agonist-L-S(═O)₂(CH₂)₂— sulphur in cysteine residue in protraction protein,
GLP-1 agonist-L-NC(═O)CH₂— sulphur in cysteine residue in protraction protein, and

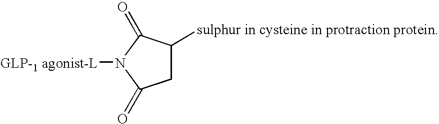

35. The compound according to claim 1, which is selected from the group consisting of S-gamma³⁴-(1-{2-[2-(2-([D-Ala⁸, Lys³⁷]-GLP-1-(7-37)amide-N^{ε37}-yl)acetyloxyethoxy)ethylcarbamoyl]ethyl}-2,5-dioxo-pyrrolidin-3-yl)Albagen 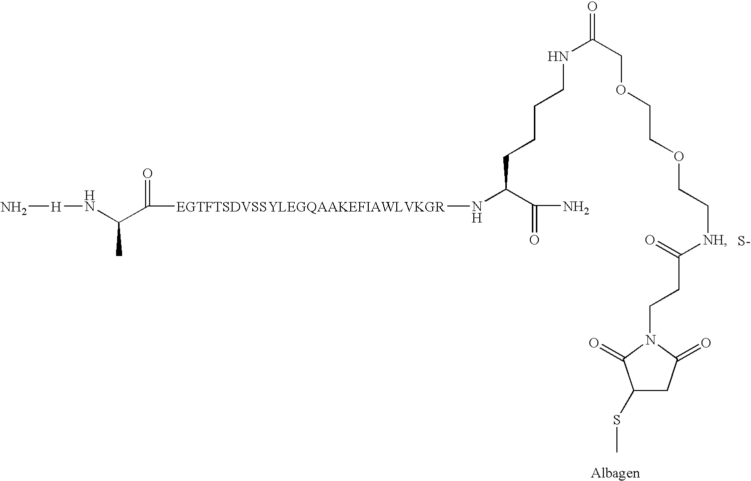
gamma³⁴-(1-{2-[2-(2-([Aib^{8,22,25}, Lys³⁷]-GLP-1-(7-37)amide-N^{ε37}-yl)acetyloxyethoxy)ethylcarbamoyl]ethyl}-2,5-dioxo-pyrrolidin-3-yl)Albagen 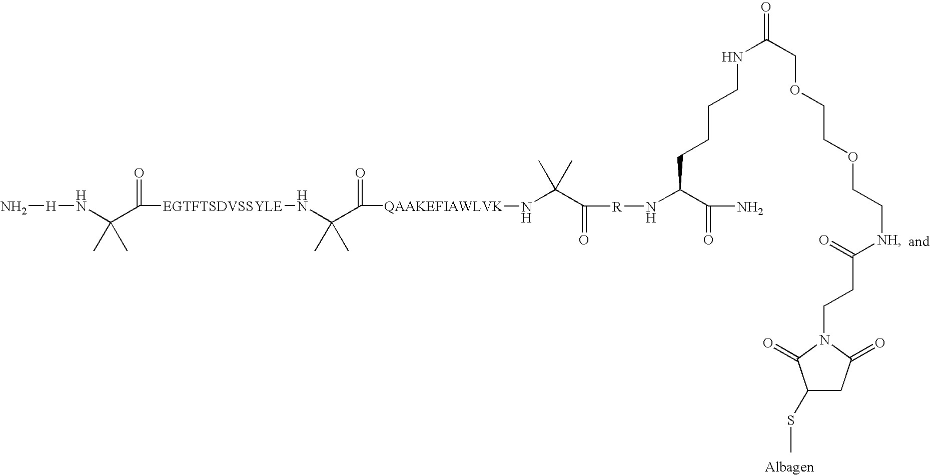
S-gamma³⁴-((1-{2-[2-(2-([Aib8,Arg26,34,Glu22,23,30]-GLP-1-(7-37))Lys amide-N^{ε}-yl)acetyloxyethoxy)ethylcarbamoyl]ethyl}-2,5-dioxo-pyrrolidin-3-yl)Albagen

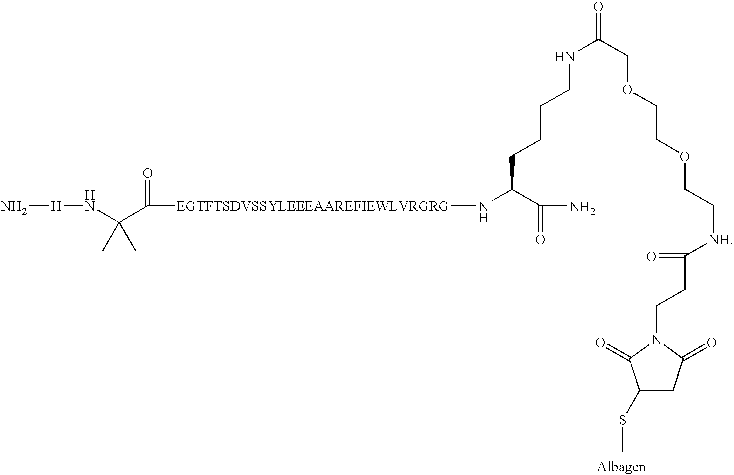

36. A compound according to claim 1, which is selected from the group consisting of
S-γ³⁴-(1-{2-[2-(2-([Lys³²]-exendin-(1-39)amide-N-ε³²-yl)acetyloxyethoxy)ethylcarbamoyl]ethyl}-2,5-dioxo-pyrrolidin-3-yl) Albumin (wherein Albumin is recombinant Albagen from New Century Pharma, i.e. recombinant HSA(2-585)),
S-γ³⁴-(1-{2-[2-(2-([Lys²⁰]-exendin-(1-39)amide-N-ε²⁰-yl)acetyloxyethoxy)ethylcarbamoyl]ethyl}-2,5-dioxo-pyrrolidin-3-yl) Albumin (wherein Albumin is recombinant Albagen from New Century Pharma, i.e. recombinant HSA(2-585)),
S-γ³⁴-(1-{2-[2-(2-([Arg¹², Lys²⁷]-exendin-(1-39)amide-N-ε²⁷-yl)acetyloxyethoxy)ethylcarbamoyl]ethyl}-2,5-dioxo-pyrrolidin-3-yl) Albumin (wherein Albumin is recombinant Albagen from New Century Pharma, i.e. recombinant HSA(2-585)), and
S-γ³⁴-(1-{2-[2-(2-([Arg^{12,27}, Lys³²]-exendin-(1-39)amide-N-ε³²-yl)acetyloxyethoxy)ethylcarbamoyl]ethyl}-2,5-dioxo-pyrrolidin-3-yl) Albumin (wherein Albumin is recombinant Albagen from New Century Pharma, i.e. recombinant HSA(2-585)).

37. A pharmaceutical composition comprising a compound according to claim 1 and a pharmaceutically acceptable preservative.

38. A pharmaceutical composition comprising a compound according to claim 1 and a pharmaceutically acceptable stabilizer.

39. The pharmaceutical composition according to claim 37 which is suited for parenteral administration.

40. The pharmaceutical composition according to claim 38 which is suited for parenteral administration.

41. A method for treating hyperglycemia, type 2 diabetes, impaired glucose tolerance, type 1 diabetes, obesity, hypertension, syndrome X, dyslipidemia, cognitive disorders, atheroschlerosis, myocardial infarction, coronary heart disease and other cardiovascular disorders, stroke, inflammatory bowel syndrome, dyspepsia and gastric ulcers, said method comprising administering to a subject in need of such treatment an effective amount of a compound according to claim 1.

42. A method for delaying disease progression in type 2 diabetes in a subject, said method comprising administering to said subject an effective amount of a compound according to claim 1.

43. A method for decreasing food intake, decreasing β-cell apoptosis, increasing β-cell function and β-cell mass, and/or for restoring glucose sensitivity to β-cells in a subject, said method comprising administering to said subject an effective amount of a compound according to claim 1.


SEQUENCE LISTING (5 sequences)

SEQ ID NO: 1 - protein, 585 aa, Homo sapiens [sequence omitted]

SEQ ID NO: 2 - protein, 31 aa, Homo sapiens
HAEGTFTSDVSSYLEGQAAKEFIAWLVKGRG

SEQ ID NO: 3 - protein, 39 aa, Heloderma suspectum
  MOD_RES (39)..(39): amidation of carboxy group
HGEGTFTSDLSKQMEEEAVRLFIETLKNGGPSSGAPPPS

SEQ ID NO: 4 - protein, 40 aa, Artificial
  Synthetic
  MISC_FEATURE (1)..(1): Xaa at position 1 is L-histidine, D-histidine, desamino-histidine, 2-amino-histidine, beta-hydroxy-histidine, homohistidine, N-alpha-acetyl-histidine, alpha-fluoromethyl-histidine, alpha-methyl-histidine,
  MISC_FEATURE (2)..(2): Xaa at position 2 is Ala, D-Ala, Gly, Val, Leu, Ile, Lys, Aib, (1-aminocyclopropyl) carboxylic acid, (1-aminocyclobutyl) carboxylic acid,(1-aminocyclopentyl) carboxylic acid, (1-aminocyclohexyl) carboxylic acid, (1-aminocycloheptyl)
  MISC_FEATURE (2)..(2): Xaa at position 2 is also  (1-aminocyclooctyl) carboxylic acid
  MISC_FEATURE (10)..(10): Xaa at position 10 is Val or Leu
  MISC_FEATURE (12)..(12): Xaa at position 12 is Ser, Lys or Arg.
  MISC_FEATURE (13)..(13): Xaa at position 13 is Tyr or Gln.
  MISC_FEATURE (14)..(14): Xaa at position 14 is Leu or Met.
  MISC_FEATURE (16)..(16): Xaa at position 16 is Gly, Glu or Aib.
  MISC_FEATURE (17)..(17): Xaa at position 17 is Gln, Glu, Lys or Arg
  MISC_FEATURE (19)..(19): Xaa at position 19 is Ala or Val
  MISC_FEATURE (20)..(20): Xaa at position 20 is Lys, Glu or Arg.
  MISC_FEATURE (21)..(21): Xaa at position 21 is Glu or Leu.
  MISC_FEATURE (24)..(24): Xaa at position 24 is Ala, Glu or Arg
  MISC_FEATURE (27)..(27): Xaa at position 27 is Val or Lys.
  MISC_FEATURE (28)..(28): Xaa at position 28 is Lys, Glu, Asn or Arg
  MISC_FEATURE (29)..(29): Xaa at position 29 is Gly or Aib.
  MISC_FEATURE (30)..(30): Xaa at position 30 is Arg, Gly or Lys
  MISC_FEATURE (31)..(31): Xaa at position 31 is Gly, Ala, Glu, Pro, Lys, or is absent.
  MISC_FEATURE (32)..(32): Xaa at position 32 is Lys, Ser, or is absent
  MISC_FEATURE (33)..(33): Xaa at position 33 is Ser, Lys, or is absent
  MISC_FEATURE (34)..(34): Xaa at position 34 is Gly, or is absent.
  MISC_FEATURE (35)..(35): Xaa at position 35 is Ala, or is absent
  MISC_FEATURE (36)..(36): Xaa at position 36 is Pro, or is absent
  MISC_FEATURE (37)..(37): Xaa at position 37 is Pro, or is absent.
  MISC_FEATURE (38)..(38): Xaa at position 38 is Pro, or is absent.
  MISC_FEATURE (39)..(39): Xaa at position 39 is Ser or is absent.
  MISC_FEATURE (40)..(40): Xaa at position 40 is absent.
XXEGTFTSDXSXXXEXXAXXXFIXWLXXXXXXXXXXXXXX

SEQ ID NO: 5 - protein, 32 aa, Artificial
  Synthetic
  MISC_FEATURE (1)..(1): Xaa at position 1 is L-histidine, D-histidine, desamino-histidine, 2-amino-histidine, beta-hydroxy-histidine, homohistidine, N-alpha-acetyl-histidine, alpha-fluoromethyl-histidine, alpha-methyl-histidine,
  MISC_FEATURE (2)..(2): Xaa at position 2 is Ala, D-Ala, Gly, Val, Leu, Ile, Lys, Aib, (1-aminocyclopropyl) carboxylic acid, (1-aminocyclobutyl) carboxylic acid,(1-aminocyclopentyl) carboxylic acid, (1-aminocyclohexyl) carboxylic acid, (1-aminocycloheptyl)
  MISC_FEATURE (2)..(2): Xaa at position 2 is also  (1-aminocyclooctyl) carboxylic acid
  MISC_FEATURE (12)..(12): Xaa at position 12 is Ser, Lys or Arg.
  MISC_FEATURE (16)..(16): Xaa at position 16 is Gly, Glu or Aib.
  MISC_FEATURE (17)..(17): Xaa at position 17 is Gln, Glu, Lys or Arg
  MISC_FEATURE (20)..(20): Xaa at position 20 is Lys, Glu or Arg.
  MISC_FEATURE (24)..(24): Xaa at position 24 is Ala, Glu or Arg
  MISC_FEATURE (28)..(28): Xaa at position 28 is Lys, Glu or Arg
  MISC_FEATURE (29)..(29): Xaa at position 29 is Gly or Aib.
  MISC_FEATURE (30)..(30): Xaa at position 30 is Arg or Lys
  MISC_FEATURE (31)..(31): Xaa at position 31 is Gly, Ala, Glu or Lys
  MISC_FEATURE (32)..(32): Xaa at position 32 is Lys or is absent
XXEGTFTSDVSXYLEXXAAXEFIXWLVXXXXX